(19) United States
(12) Patent Application Publication
(10) Pub. No.: US 2008/0103815 A1
(43) Pub. Date: May 1, 2008

(54) System and method for estimating cost of medical treatment

(75) Inventor: Ajmal Mirza Muhammad Beg, The Gap (AU)

Correspondence Address: Fountainhead Law Group P.C., 900 LAFAYETTE STREET, SUITE 509, SANTA CLARA, CA, 95050, US

(73) Assignee: SAP AG, Walldorf (DE)

(21) Appl. No.: 11/590,331

(22) Filed: Oct. 31, 2006

(51) Int. Cl. G06Q 10/00; G06Q 40/00; A61B 5/00

(52) U.S. Cl. 705/2; 600/300; 705/4

(57) ABSTRACT

In one embodiment, a method for estimating total cost of medical treatment comprises (a) receiving medical treatment data including medical facility location data, treatment cost data, and follow-up treatment data corresponding to at least one medical treatment provided by a plurality of medical facilities; (b) receiving accommodation data including accommodation location data and accommodation cost data for at least one accommodation in proximity to at least one medical facility of the plurality of medical facilities; and (c) providing the medial treatment cost data and the accommodation cost data to a user for estimating a total cost of the medical treatment. In some embodiments, the method further comprises calculating the total treatment cost from the accommodation cost data and the follow-up treatment data, and providing the treatment data and the accommodation cost data comprises providing the calculated total treatment cost.

### BACKGROUND

The present invention relates to health care information systems, and in particular, to systems and methods for estimating total cost of medical treatment.

Unless otherwise indicated herein, the approaches described in this section are not prior art to the claims in this application and are not admitted to be prior art by inclusion in this section.

When a patient needs a medical treatment requiring stay in a hospital, he or she may wish to select a hospital that provides high quality medical service at a reasonable cost. Using available treatment quality data provided by hospitals and agencies in each state, the patient or family doctor can compare the quality and costs of different procedures at different hospitals or other medical facilities.

For example, the United States Department of Health and Human Services' Agency for Healthcare Research and Quality (AHRQ) provides an array of health care decision making and research tools that can be used by patients, program managers, researchers, and others at the Federal, State and local levels. One of these tools is the AHRQ Quality Indicators (QIs), which use hospital administrative data to highlight potential quality concerns, identify areas that need further study and investigation, and track changes over time. The AHRQ QIs are comprised of the Inpatient Quality Indicators (IQIs), Prevention Quality Indicators (PQIs), the Patient Safety Indicators (PSIs), and the Pediatric Quality Indicators (PDIs).

The Inpatient Quality Indicators (IQIs) are a set of measures that provide a perspective on hospital quality of care using hospital administrative data. These indicators reflect quality of care inside hospitals and include inpatient mortality for certain procedures and medical conditions; utilization of procedures for which there are questions of overuse, underuse, and misuse; and volume of procedures for which there is some evidence that a higher volume of procedures is associated with lower mortality.

The Patient Safety Indicators (PSIs) are a set of indicators providing information on potential inhospital complications and adverse events following surgeries, procedures, and childbirth. In particular, PSI's provide a measure of complications that patients experienced that might have been potentially preventable for patients who received their initial care and experienced a complication within the same hospitalization.

The Prevention Quality Indicators (PQIs) are a set of measures that can be used with hospital inpatient discharge data to identify quality of care for “ambulatory care-sensitive conditions.” These are conditions for which good outpatient care can potentially prevent the need for hospitalization or for which early intervention can prevent complications or more severe disease.

The Pediatric Quality Indicators (PDIs) are a set of measures that can be used with hospital inpatient discharge data to provide a perspective on the quality of pediatric healthcare. Specifically, PDIs screen for problems that pediatric patients experience as a result of exposure to the healthcare system and that may be amenable to prevention by changes at the system or provider level.

Additional details regarding the AHRQ quality indicators and tools may be found, for example, in Sheryl M. Davies et al., Refinement of the HCUP Quality Indicators, Agency for Healthcare Research and Quality Publication No. 01-0035, May 2001; and Kathryn M. McDonald et al., Measures of Patient Safety Based on Hospital Administrative Data: The Patient Safety Indicators, AHRQ Publication No. 02-0038, August 2002.

Some government entities have created web-based systems that enables potential patients to compare treatment quality data from different medical facilities. Such systems may use treatment quality indicators and tools from AHRQ and related classification tools from 3M Corporation. Such systems may provide data and information to help patients compare quality and cost of care for various treatments or procedures performed at different hospitals and ambulatory surgery centers.

Such systems may utilizes standard hospital performance measures to provide quantitative information to prospective patients. These hospital performance measures include, for example, average length of stay, average charge (cost), patient safety indicators, complication index, and mortality rate.

The Average Length of Stay is one of the hospital quality or performance indicators which can help determine the relative efficiency and cost of a procedure at a particular hospital compared to other hospitals. In particular, the Average Length of Stay is the typical number of days a patient stayed in the hospital for a particular condition or procedure. For a fair comparison between hospitals, the information may be risk adjusted to take into account that some hospitals take care of patients who are sicker and require more treatment or resources than the “average” patient. Average length of stay provides an idea of how long one might expect to stay in the hospital as determined by the attending physician. The average length of stay might reflect the efficiency of care provided by a hospital.

The Average Charge is the average amount that the hospital billed for patients discharged from the hospital, who had that particular condition or procedure. This Average Charge measure may be used to learn the typical charges for a condition or procedure. The hospital charge may not reflect the actual cost or the amount paid for the care, which ultimately depends on the type of insurance coverage, co-payments and/or deductibles, or if a patient is uninsured, whether that patient qualifies for discounts under the hospital's discount or charity policies. Since charges can be impacted by patient differences, the charges may be risk adjusted to account for these patient differences.

Patient Safety Indicators are a set of measures that can provide one view on patient safety. They provide a measure of complications that patients experienced that might have been potentially preventable for patients who received their initial care and experienced a complication within the same hospitalization.

The Complication Index is a combination of five patient safety indicators developed by the Wisconsin Collaborative for Healthcare Quality and the National Association of Health Data Organizations (NAHDO), and indicates a rate of problems or complications that patients experienced while in the hospital that might have been preventable.

The Mortality Rate reports the percentage of patients who died at a hospital after undergoing a specific type of surgery or while being treated for a specific condition. Mortality Rate data may be based, for example, on the Inpatient Quality Indicators developed by the U.S. Dept. of Health and Human Services' AHRQ.

Readmission Rate provides an indication of effectiveness of a hospital's treatment. For example, a low readmission rate at a hospital may indicate that the hospital is effective in caring for a patient's problems so that additional hospital care is not needed in most cases. A low rate may also indicate the care given to a patient after they leave the hospital is effective in preventing a return to the hospital.

While existing systems attempt to provide comprehensive quality and cost comparisons, they do not take into account the fact that the total length of a treatment often exceeds the actual length of stay in the hospital. For example, for a surgery, a patient may spend five days in the hospital, and then over the course of the next 15 days the patient may need to undergo regular follow-up treatments requiring visits to the hospital every second day. Depending upon the location of the hospital with respect to the residence of the patient, the patient may be required to pay for lodging or other accommodations from where the patient can easily visit the hospital during the follow-up treatment period. In some cases, the patient may be able to stay in an accommodation that does not incur substantial cost, such as the home of a friend or relative near the medical facility. In other cases, however, the patient may need to stay at a hotel, apartment or other rental accommodation near the hospital.

The average length of stay figure does not provide the patient with the total cost of the treatment including the hospitalization cost and the cost of paid accommodation near the hospital when the patient must stay in a paid accommodation after discharge from the hospital. Thus, there is a need for improved systems and methods for estimating total cost of medical treatment to enable patients to make informed decisions regarding medical treatment facilities. The present invention solves these and other problems by providing systems and methods of estimating total cost of medical treatment.

### SUMMARY

Embodiments of the present invention improve comparisons of quality and total cost of medical treatments at different hospitals or other medical facilities.

In one embodiment, a method of providing estimated total cost of a medical treatment, comprises: (a) receiving medical treatment data including medical facility location data, treatment cost data, and follow-up treatment data corresponding to at least one medical treatment provided by a plurality of medical facilities; (b) receiving accommodation data including accommodation location data and accommodation cost data for at least one accommodation in proximity to at least one medical facility of the plurality of medical facilities; and (c) providing the medial treatment cost data and the accommodation cost data to a user for estimating a total cost of the medical treatment.

In some embodiments, the method may further comprise calculating a total treatment cost from the accommodation cost data and the follow-up treatment data, wherein providing the treatment cost data and the accommodation cost data comprises providing the calculated total treatment cost to the user. In some embodiments, the medical treatment data is received from a plurality of medical providers over a network, and further comprises medial facility location data, treatment cost data, and follow-up treatment data corresponding to a plurality of medical treatments provided by the plurality of medical facilities.

In one embodiment, A computer readable storage medium having a computer program mechanism embedded therein comprises instructions for (a) receiving medical treatment data comprising treatment cost information corresponding to a plurality of medical treatments provided by a plurality of medical facilities; (b) receiving accommodation data comprising accommodation location data and accommodation price data for a plurality of accommodations; and (c) analyzing the medical treatment data and the accommodation data to generate a result comprising the treatment cost data from at least one medical facility and the accommodation cost data corresponding an accommodation located in proximity to the medical facility. The medical treatment data may further comprise hospital length of stay data and/or follow up treatment data.

In some embodiments, the storage medium also comprises instructions for providing the result to a client computer over a network in response to a query from the client computer, for calculating a total treatment cost for a treatment provided by the at least one medical facility from the accommodation cost data and the follow-up treatment data, and/or for storing the medical treatment data and the accommodation data in a database. In some embodiments, the storage medium further comprises instructions for formatting the treatment quality data into a uniform format before storing. In some embodiments, the medical treatment data further comprises any of a medical specialty identifier, a description of medical procedures under each specialty, success rates for different procedures, infection rates, a complication index, a readmission rate, an Inpatient Quality Indicator, a Prevention Quality Indicator, a Patient Safety Indicator, or a Pediatric Quality Indicator.

In one embodiment, a medical treatment cost analysis server collects treatment quality and cost data, including costs associated with the length of stay in the hospital and number of days that the patient must remain in the proximity of the hospital for follow-up visits (also referred to as follow-up data). The server calculates treatment costs associated with both types of treatment days. This allows the patient to estimate the approximate total cost of the treatment, which can be taken into account along with treatment and facility quality data to make an informed decision.

In another embodiment the present invention includes a system for estimating total cost of medical treatment, comprising (a) a central processing unit; (b) a data collection module communicating with the central processing unit, said data collection module for communication over a network with at least one medical treatment data provider to collect medical treatment data corresponding to a plurality of medical treatments performed at a plurality of medical facilities and accommodation data corresponding to a plurality of accommodations located in proximity to one or more of the plurality of medical facilities; (c) a database communicating with processor for receiving data from the data collection module, said database storing collected medical treatment data and collected accommodation data; and (d) a query engine communicating with the central processing unit for receiving a medical treatment search query from a client computer and returning to the client a query result comprising the medical treatment data and corresponding accommodation data.

In some embodiments, the accommodation data comprises accommodation price data corresponding to accommodations located in proximity to one or more of the plurality of medical facilities, and the medical treatment data comprises a follow-up treatment duration parameter. The query result may further comprise a total treatment cost parameter calculated from the accommodation price data and the follow-up treatment duration parameter. In some embodiments, the system may further include a data processing module for formatting the medical treatment data into a standardized format, and a data analysis module for analyzing the medical treatment data and the accommodation data and a generating a report comprising the medical treatment data and the accommodation data.

The following detailed description and accompanying drawings provide a better understanding of the nature and advantages of the present invention.

### BRIEF DESCRIPTION OF THE DRAWINGS

FIG. 1 is a schematic functional diagram of a medical treatment cost analysis system according to one embodiment of the present invention.

FIG. 2 is a schematic diagram of a medical treatment cost analysis system and server according to another embodiment of the present invention.

FIG. 3A illustrates a method of analyzing medical treatment costs using the server of FIG. 2.

FIG. 3B is a flowchart describing the method of FIG. 3A.

FIG. 4 is an illustration of a user interface depicting a treatment quality and cost query user form according to one embodiment of the present invention.

FIG. 5 is an illustration of a user interface depicting a query search result form according to one embodiment of the present invention.

FIG. 6 is an illustration of a user interface screen for providing query result sorting instructions according to one embodiment of the present invention.

FIG. 7 is a schematic diagram of a data model according to one embodiment of the present invention.

### DETAILED DESCRIPTION

Described herein are apparatus and techniques for estimating total cost of medical treatments. In the following description, for purposes of explanation, numerous examples and specific details are set forth in order to provide a thorough understanding of the present invention. It will be evident, however, to one skilled in the art that the present invention as defined by the claims may include some or all of the features in these examples alone or in combination with other features described below, and may further include modifications and equivalents of the features and concepts described herein.

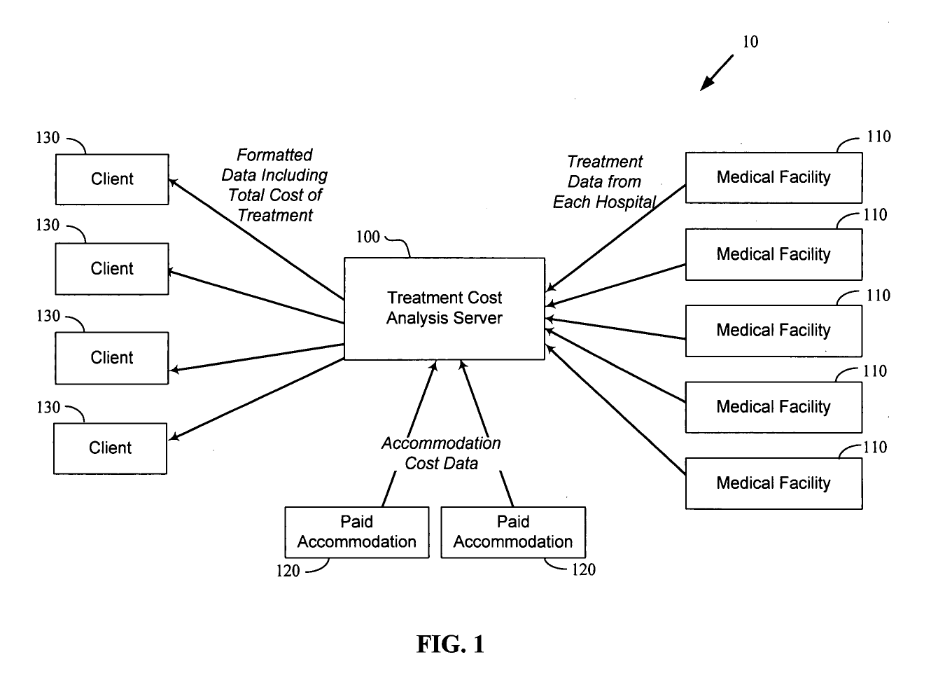

FIG. 1 is a schematic functional diagram of a medical treatment cost analysis system 10 according to one embodiment of the present invention. System 10 includes a treatment cost analysis server 100 which acts as a hub between medical facilities 110, external accommodation providers 120, and clients 130, or users of the medical facilities 110. Server 100 receives or retrieves (generally referred to herein as “retrieves”) treatment data from each of a plurality of medical facilities, and/or from one or more providers of such treatment data (e.g., public or private databases comprising treatment data corresponding to one or more medical facilities).

As used herein, the treatment cost analysis server 100 may also be referred to as a treatment quality data server. Similarly, the terms “treatment quality data” or “treatment data” may be used broadly herein to include various types of treatment quality and cost parameters, including for example average cost, length of stay, number and frequency of follow-up visits, success rates, mortality rates, and/or other Quality Indicators or performance measures for different procedures at different medical facilities as described in the Background section above. The treatment data also preferably includes identification, location and/or other information specific to each medical facility and/or medical unit. Treatment data related to the number, frequency and/or total duration of follow-up treatment visits, e.g., outpatient care or other visits required after release from the medical facility, is referred to herein as “follow-up treatment data”.

Server 100 also retrieves accommodation data from accommodation providers 120, e.g., hotels, motels, apartments, etc., which are located in proximity to one or more of the medical facilities. In some embodiments, data for each accommodation provider may be retrieved directly form that provider, or data for a number of accommodations may be retrieved from a data provider or database having information corresponding to a number of different accommodations (e.g., a hotel or motel chain or group, or a reservation agent system). The accommodation data may include, for example, provider name, type, location, cost, ranking and/or parameters related to each accommodation. A proximity range, e.g., 1 mile, 5 miles, 10 miles, 20 miles or more, may be pre-defined by system 100 and/or may be defined by the user or client 130 in a query.

In some embodiments, the treatment data and accommodation data are retrieved and at least partially processed by the server 100 prior to receiving a search query from a client 130. For example, treatment data received from a number of different medical facilities and regarding a number of different medical procedures may need to be standardized or otherwise processed into a uniform format for efficient storage and use by server and/or presentation to a user 130. The formatted data provided to the clients 130 may also include calculations, estimations, scores, derivations, indexes, or other derived data to assist the user in making an informed decision regarding a desired medical treatment. For example, the formatted treatment and accommodation data provided by server 100 may include estimates of total cost of treatment, e.g., by multiplying the duration of follow up visits for each treatment type at each hospital by the cost of an accommodation in proximity to the hospital. Other calculations may be employed to derive any desired estimates or other parameters for comparison by a user. Similarly, other cost data may be taken into account, e.g., whether facilities accept particular medical insurance plans, or frequency, distance and cost of transportation.

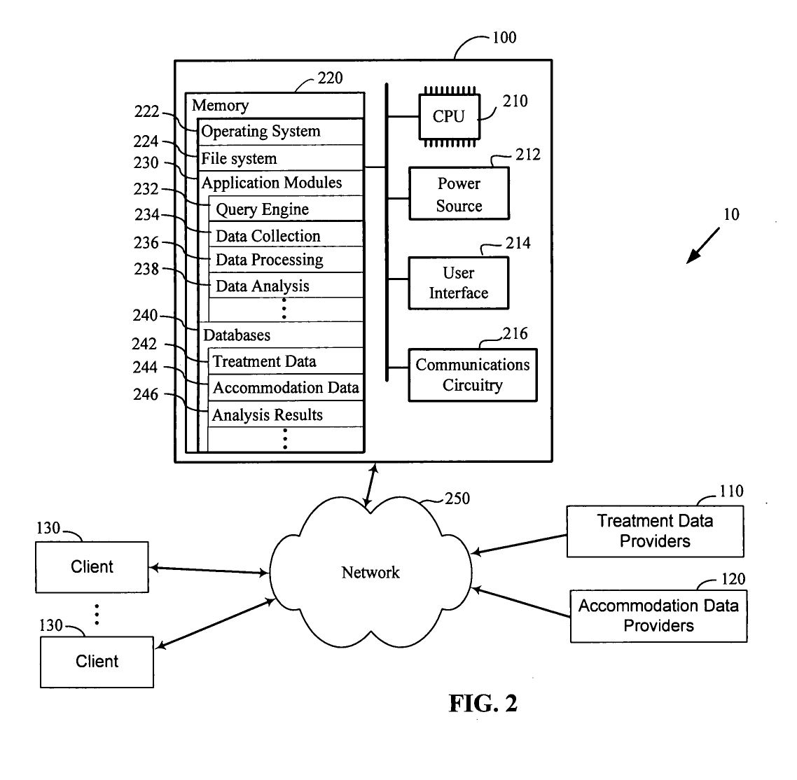

FIG. 2 details an exemplary system 10 that supports the functionality described above and detailed in sections below. System 10 comprises a medical treatment quality and cost server system 100 in communication over a network 250 with medical treatment data providers 110, accommodation providers 120, and clients 130 as described with respect to FIG. 1.

Network 250 can comprise one or more wired or wireless networks, e.g., the internet or other wide area network (WAN), one or more local area networks, wired or wireless telephone networks (e.g., a telephone network, a voice over integrated packet (VOIP) network, or a GSM, PCS, mobitex, CDMA, TDMA or other network for voice and/or data communications).

In some embodiments, system 100 may comprise a central processing unit 210, a power source 212, communications circuitry 216, a bus 218, and at least one memory 220. Memory 130 may comprise volatile and non-volatile storage units, for example hard disk drives, random-access memory (RAM), read-only memory (ROM), flash memory and the like. In preferred embodiments, memory 220 comprises high-speed RAM for storing system control programs, data, and application programs, e.g., programs and data loaded from non-volatile storage. System 100 optionally includes a user interface 214, which may comprise one or more input devices, e.g., keyboard, key pad, mouse, scroll wheel, and the like, and a display or other output device. A network interface card or other communication circuitry 216 may provide for connection to any wired or wireless communications network 250, which may include the internet and/or any other wide area network. Internal bus 218 provides for interconnection of the aforementioned elements of system 100.

Operation of system 100 is controlled primarily by an operating system 222, which is executed by central processing unit 210. Operating system 222 can be stored in system memory 220. In addition to operating system 222, in a typical implementation system memory 220 may include a file system 224 for controlling access to the various files and data structures used by the present invention, one or more application modules 230, and one or more databases or data modules 240.

The applications modules 230 may comprise one or more of the following:
a query engine 232 for receiving and responding to search queries from a client 130;
a data collection module 234 for collecting data from medical facilities, accommodation providers, and/or other data providers;
a data processing module 236 for formatting or otherwise processing the collected data; and
a data analysis module 238 for analyzing the collected data and/or creating reports of compiled data.

The applications module 230 may comprise other application modules, for example a web server or other system or application for hosting and/or managing web page files and other content associated with a web site related to the service, preferably the web site and/or pages are configured for easy accessibility by a WAP-enabled mobile device.

The one or more data modules 150 may include any number of data files, tables and/or other structures, including for example, treatment data 242, accommodation data 244 and analysis results 246. Treatment data 242 may include various types of medical treatment quality and cost parameters associated with different treatments or procedures performed by at each medical facility, e.g., treatment cost, length of stay, number, frequency and/or duration of follow-up visits, treatment success rates, mortality rates, and/or other Quality Indicators or performance measures for different procedures at different medical facilities. Accommodation data 244 may include, for example, provider name, type, location, cost, ranking and/or parameters related to each accommodation. Analysis results 246 may include, for example, total cost calculations for each medical procedure at each facility, including costs associated with paid accommodations which may be required due to the location of the facility and the required follow-up treatment regimen.

In some embodiments, one or more search parameters or fields (e.g., distance between hospital and an accommodation, accommodation class, accommodation cost, and/or any other desired search parameter) may be assigned a weight or weighting factor. In particular embodiments, such weight or weighting factor may be assigned or changed by the end user, and the search results may be returned ranked according to a function that uses the user assigned weights for the selected search fields.

In some embodiments, each of the aforementioned data structures stored or accessible to system 100 are single data structures. In other embodiments, any or all such data structures may comprise a plurality of data structures (e.g., databases, files, archives) that may or may not all be stored on system 100. For example, in some embodiments, data modules 240 comprise a plurality of structured and/or unstructured data records that are stored on computer 100 and/or on computers that are addressable by computer 100 across the network 250.

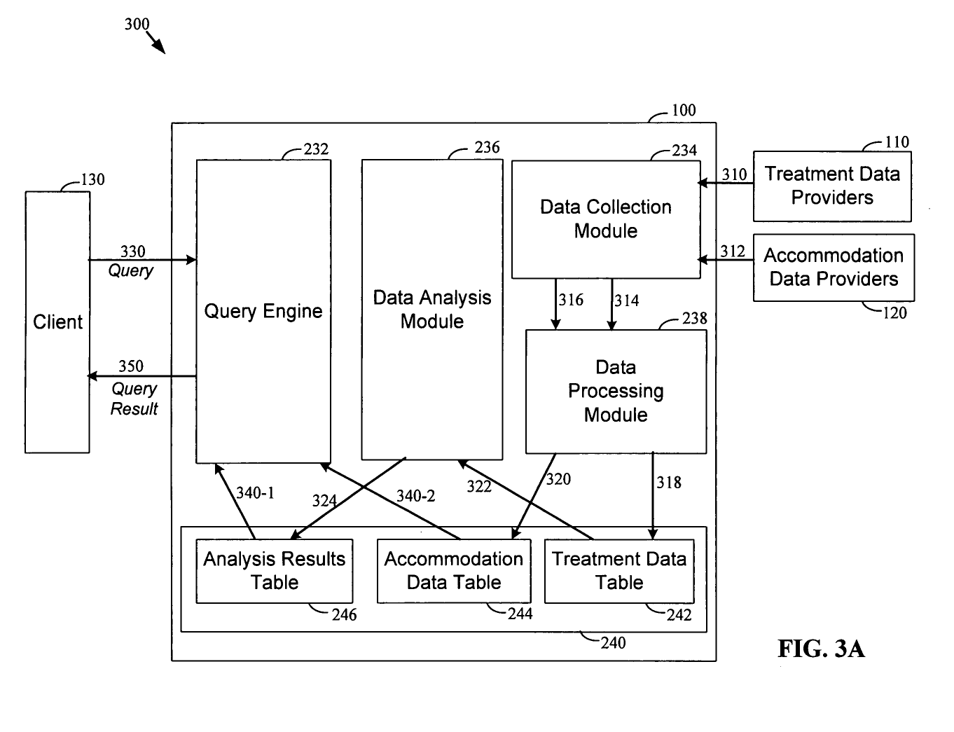

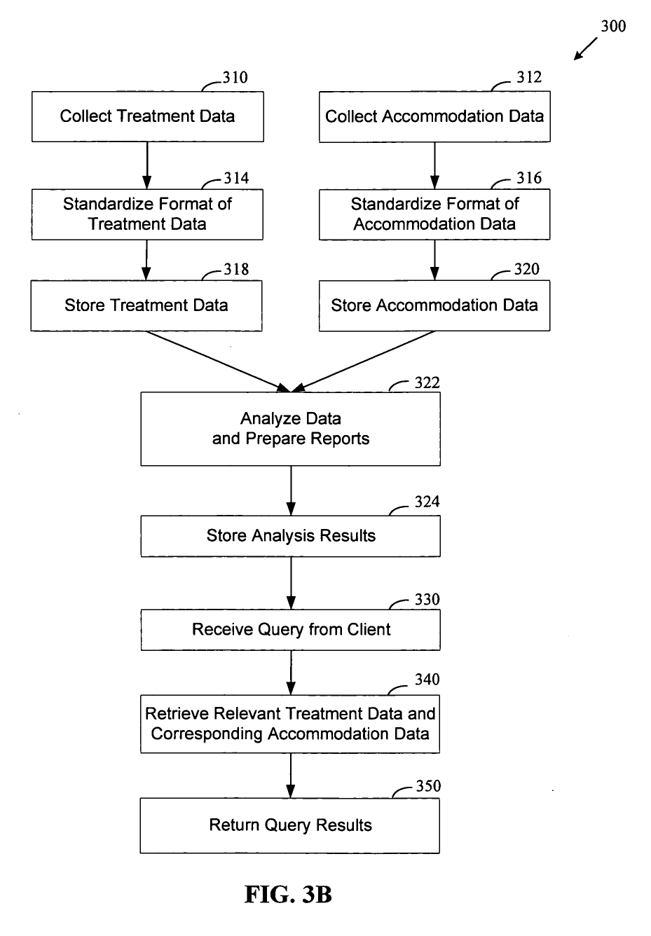

FIG. 3A is a functional diagram depicting the various software modules, including data collection module 234, data processing module 238, data analysis module 236, query engine 232 and data modules 240, which interact with external parties 110, 120 and 130. FIG. 3B illustrates a method of analyzing medical treatment costs using the data modules depicted in FIG. 3A. In steps 310 and 312, data collection module 234 collects treatment/procedure data from treatment data providers 110 and accommodation providers 312. The collecting 310 and 312 may be initiated by server 100 or by providers 110, 120, and may or may not occur according to a predetermined schedule. Treatment data collected in 310 may be processed into a standardized or uniform format in 314 before being stored at 318 in treatment data table or other data structure 242 of database 240. Similarly, accommodation data collected in 312 may be processed into a standardized or uniform format in 316 before being stored at 320 in accommodation data table 244.

After treatment and accommodation data are stored, they may be analyzed and/or further processed in 322, e.g., by data analysis module 236. In particular embodiments, for example, analysis module 236 prepares reports containing statistical information such as average days of stay in a hospital and corresponding external accommodation options and associated parameters and costs. Results and reports compiled from the analyses performed in 322 are stored at 324 in analysis results module 246 until needed. In some embodiments, the results and reports are compiled and stored according to a schedule stored in system 100. Alternatively, such analysis may be performed in response to the presence of new data, or in accordance with instructions from an administrator or user.

In one embodiment, the user or client 130 sends a query 330 to query engine 232 to perform a search for comparing treatment and cost parameters of different medical facilities for a desired type of treatment. The query may be submitted by client 130 to system 100, for example by using a fillable and/or selectable form, e.g., JAVA, XML, HTML, or any other type of interactive data entry form, received or hosted by server 100 or another computer and accessible to client 130 over network 250.

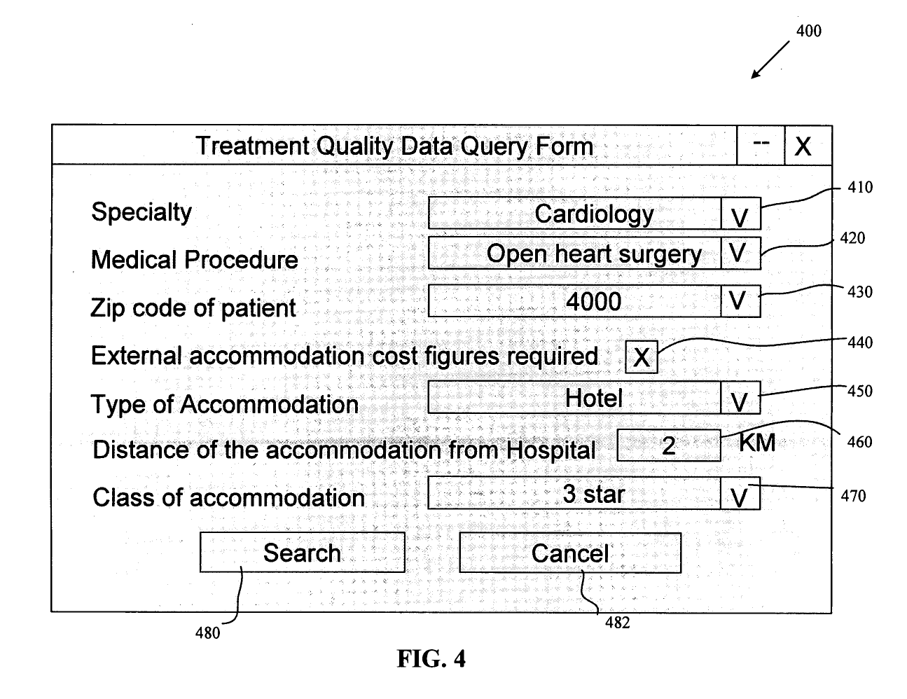

For example, a treatment quality data query interface form 400 as illustrated in FIG. 4 may be completed and submitted by client 130 to query the server 100. In this example, form 400 includes a number of different selectable fields, including pull-down menus for specialty 410, medical procedure 420, zip code of patient 430, type of accommodation 450, and class of accommodation 470. A fillable field 460 may provide distance of the accommodation from the hospital. Other fields may include, for example, a selectable or toggle feature to indicating whether the user desires to receive external accommodation cost figures. Other features may include buttons or other interface features for submitting the search query 480 and cancelling query 482. One skilled in the art will appreciate that various other menu fields or parameters may be used, and other entry modes (e.g., pulldown menus, fillable fields, checkboxes, etc.) may be used for each field.

When query engine 232 receives a treatment quality data query from a client 130 in step 330, query engine 232 retrieves the relevant treatment quality data from analysis results table 246 in 340-1 and corresponding accommodation cost information from table 244 in 340-2, and compiles the retrieved data for presentation to the user. In one embodiment, system 100 returns in step 350 a query search result report that includes data corresponding to the selected parameters and fields submitted by the user 130 on in form 400.

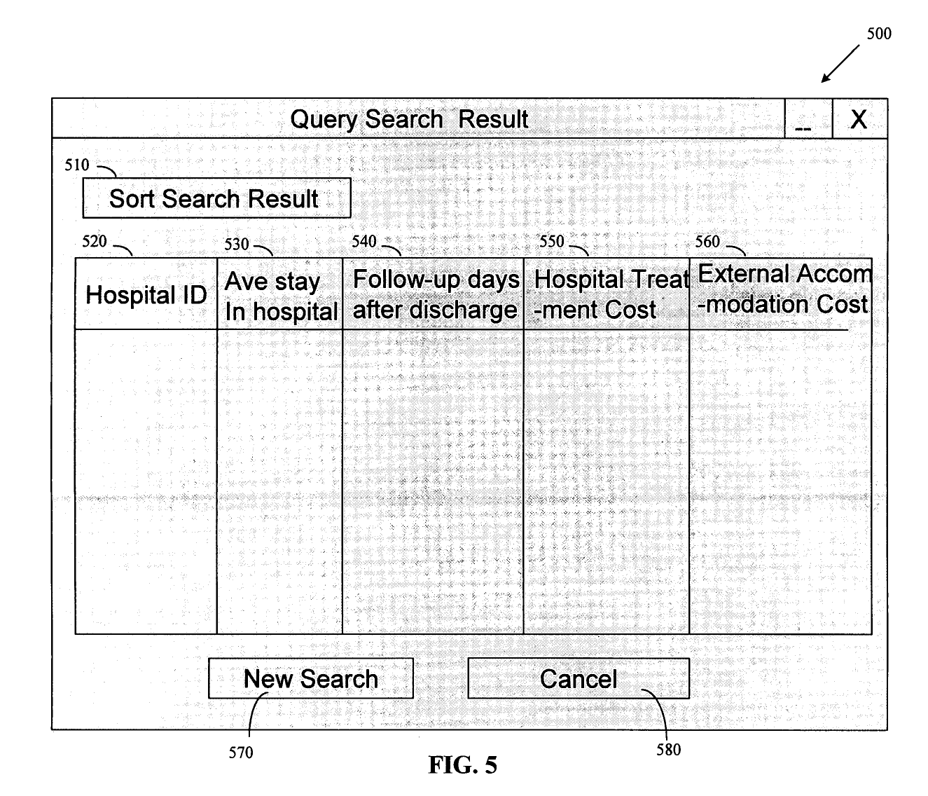

For example, FIG. 5 illustrates a sample query search result form 500, including data corresponding to hospital ID 520, average stay in hospital 530, number of follow up days after discharge 540, hospital treatment cost 550, and external accommodation cost 560 for a particular treatment. Hospital treatment cost 550 may include the cost of follow up visits in addition to in-hospital procedure costs. In other embodiments, hospital treatment cost 550 may be displayed as divided into two or more types of charge components, e.g., one for in-hospital charges and the other for outpatient costs. Of course, various other types of data, calculations, scores, parameters, and the like may be included in the search results, depending upon the desired application or query. The search form may include any number and type of desired control features, e.g., a new search button 570 and a cancel button 580.

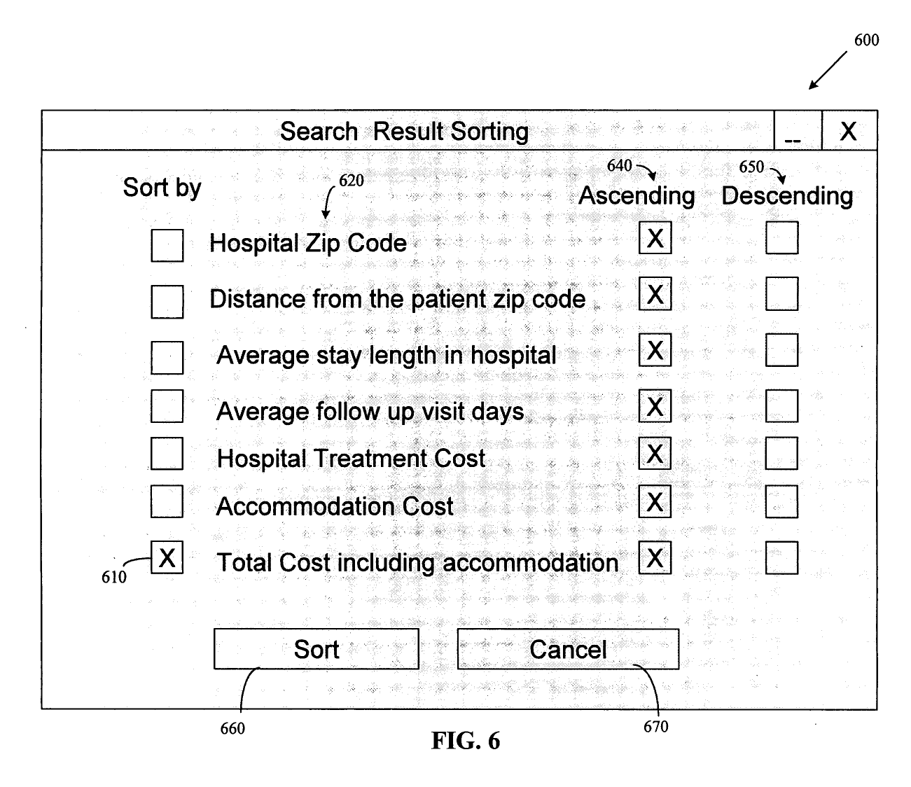

In some embodiments, search results may be filtered or sorted in accordance with default parameters or as desired by the user, for example as illustrated in FIG. 6. For example, selection of a sort button 510 or other similar software input may activate and return a sorting screen 600 or other interface for selecting desired sort parameters 620. In this example, a user has activated a check box 610 corresponding to a request to sort the search result data by the total cost of treatment including accommodation cost 620-1. Other boxes or features may be provided, for example, to select whether parameters are displayed in ascending 640 or descending 650 order. A Sort button 660 or other feature may be selected to initiate the sort function and return a sorted search result form 500, or a cancel button 670 may be selected to cancel the sort function.

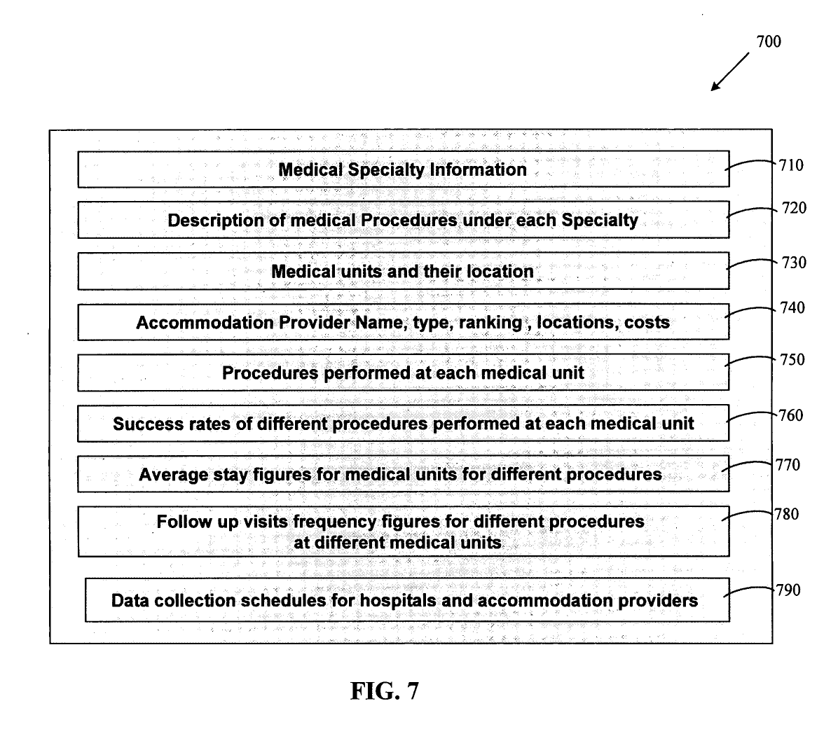

Referring now to FIG. 7, a sample data model 700 shows an example of some of the various types of treatment and accommodation data that may be processed, stored and presented by system 100. The data may be organized in tables, files, hierarchies, relational structures, or any other data structures or formats. For example, medical specialty information 710 and descriptions of medical procedures under each specialty 720 are provided, as well as medical units and their respective locations 730, procedures performed at each medical unit 750, success rates of different procedures performed at each medical unit 760. Average length of stay data for different procedures at different medical units 770 and follow up duration and frequency data 780 for different procedures at different medical units is also stored. With regard to accommodation data, parameters may include accommodation provider, name, type, ranking, locations, costs, etc.

The above description illustrates various embodiments of the present invention along with examples of how aspects of the present invention may be implemented. The above examples and embodiments should not be deemed to be the only embodiments, and are presented to illustrate the flexibility and advantages of the present invention as defined by the following claims. Based on the above disclosure and the following claims, other arrangements, embodiments, implementations and equivalents will be evident to those skilled in the art and may be employed without departing from the spirit and scope of the invention as defined by the claims.

What is claimed is:

1. A method of providing estimated total cost of a medical treatment, comprising:
receiving medical treatment data, said medical treatment data comprising medical facility location data, treatment cost data, and follow-up treatment data corresponding to at least one medical treatment provided by a plurality of medical facilities;
receiving accommodation data, said accommodation data comprising accommodation location data and accommodation cost data for at least one accommodation in proximity to at least one medical facility of the plurality of medical facilities; and
providing the medical treatment cost data and the accommodation cost data to a user for estimating a total cost of the medical treatment.

2. The method of claim 1 further comprising calculating a total treatment cost from the accommodation cost data and the follow-up treatment data, wherein providing the treatment cost data and the accommodation cost data comprises providing the calculated total treatment cost to the user.

3. The method of claim 1, wherein receiving medical treatment data comprises receiving the medical treatment data from a plurality of medical providers over a network.

4. The method of claim 3, wherein the medical treatment data further comprises medical facility location data, treatment cost data, and follow-up treatment data corresponding to a plurality of medical treatments provided by the plurality of medical facilities.

5. The method of claim 4, wherein the accommodation data comprises location data and cost data corresponding to a plurality of accommodations.

6. The method of claim 5, further comprising storing the medical treatment data and the accommodation data prior to providing the medical treatment data and the accommodation cost data to the user.

7. The method of claim 6, further comprising formatting the medical treatment data retrieved from the plurality of medical providers before storing the medical treatment data.

8. The method of claim 1, wherein said medical treatment data further comprises any of a length of stay indicator, a medical specialty identifier, a medical procedure descriptor under each specialty, a success rate indicator, an infection rate indicator, a complication index, a readmission rate indicator, an Inpatient Quality Indicator, a Prevention Quality Indicator, a Patient Safety Indicator, or a Pediatric Quality Indicator.

9. The method of claim 1, further comprising receiving a treatment search query from the user, and wherein providing the treatment cost data and the accommodation cost data to the user comprises returning a query search result including the medical treatment data and the accommodation data corresponding to search parameters in the search query.

10. A computer readable storage medium having a computer program mechanism embedded therein, said computer program mechanism comprising instructions for:
receiving medical treatment data comprising treatment cost information corresponding to a plurality of medical treatments provided by a plurality of medical facilities;
receiving accommodation data comprising accommodation location data and accommodation price data for a plurality of accommodations;
analyzing the medical treatment data and the accommodation data to generate a result comprising the treatment cost data from at least one medical facility and the accommodation cost data corresponding an accommodation located in proximity to the medical facility.

11. The computer readable storage medium of claim 10, further comprising instructions for providing the result to a client computer over a network in response to a query from the client computer.

12. The computer readable storage medium of claim 10, wherein the medical treatment data further comprises hospital length of stay data and follow-up treatment data.

13. The computer readable storage medium of claim 12 further comprising instructions for calculating a total treatment cost for a treatment provided by the at least one medical facility from the accommodation cost data and the follow-up treatment data.

14. The computer readable storage medium of claim 10, further comprising instructions for storing the medical treatment data and the accommodation data in a database.

15. The computer readable storage medium of claim 14, further comprising instructions for formatting the treatment quality data into a uniform format before said storing.

16. The computer readable storage medium of claim 10, wherein the medical treatment data further comprises any of a medical specialty identifier, a description of medical procedures under each specialty, success rates for different procedures, infection rates, a complication index, a readmission rate, an Inpatient Quality Indicator, a Prevention Quality Indicator, a Patient Safety Indicator, or a Pediatric Quality Indicator.

17. A system for providing estimated total cost of a medical treatment, comprising:
a central processing unit;
data collection module communicating with the central processing unit, said data collection module for communication over a network with at least one medical treatment data provider to collect (a) medical treatment data corresponding to a plurality of medical treatments performed at a plurality of medical facilities, and (b) accommodation data corresponding to a plurality of accommodations located in proximity to one or more of the plurality of medical facilities;
a database communicating with processor for receiving data from the data collection module, said database storing collected medical treatment data and collected accommodation data; and
a query engine communicating with the central processing unit for receiving a medical treatment search query from a client computer and returning to the client a query result comprising the medical treatment data and corresponding accommodation data.

18. The system of claim 17, wherein the accommodation data comprises accommodation price data corresponding to accommodations located in proximity to one or more of the plurality of medical facilities, and the medical treatment data comprises a follow-up treatment duration parameter.

19. The system of claim 18, wherein the query result further comprises a total treatment cost parameter calculated from the accommodation price data and the follow-up treatment duration parameter.

20. The system of claim 17, further comprising:
a data processing module for formatting the medical treatment data into a standardized format; and
a data analysis module for analyzing the medical treatment data and the accommodation data and a generating a report comprising the medical treatment data and the accommodation data.
